(19) United States
(12) Patent Application Publication
(10) Pub. No.: US 2026/0069240 A1
(43) Pub. Date: Mar. 12, 2026

(54) ULTRASOUND IMAGING APPARATUS AND METHODS FOR PRESENTING LESION DISTRIBUTION

(71) Applicant: SHENZHEN MINDRAY BIO-MEDICAL ELECTRONICS CO., LTD., Shenzhen (CN)

(72) Inventors: Wenhui CHEN, Shenzhen (CN); Yaoxian ZOU, Shenzhen (CN); Muqing LIN, Shenzhen (CN)

(73) Assignee: SHENZHEN MINDRAY BIO-MEDICAL ELECTRONICS CO., LTD., Shenzhen (CN)

(21) Appl. No.: 19/323,416

(22) Filed: Sep. 9, 2025

(30) Foreign Application Priority Data
Sep. 9, 2024 (CN) 202411260013.2

(51) Int. Cl. A61B 8/08; A61B 8/00

(52) U.S. Cl. CPC A61B 8/085; A61B 8/4444; A61B 8/465

(57) ABSTRACT

Disclosed are an ultrasound imaging apparatus and a method for presenting lesion distribution, comprising: obtaining volumetric data and identifying target tissue and target lesion therefrom to obtain three-dimensional contours of the target tissue and the target lesion, thereby generating a three-dimensional schematic diagram based on the two contours. The schematic diagram represents the morphology of the target tissue and the morphology and location of the target lesion in the target tissue, facilitating intuitive spatial comprehension of lesion distribution, surpassing conventional ultrasound images in interpretability, thereby enhancing clinical workflow efficiency. Additionally, a two-dimensional schematic diagram corresponding to at least one target section, comprising a target tissue graphic representing the morphology of the target tissue and a target lesion graphic representing the morphology and location of the target lesion, can be generated based on the three-dimensional contours of the target tissue and lesion, allowing users to understand the lesion distribution.

### CROSS-REFERENCE TO RELATED APPLICATION

The present application claims priority to Chinese Patent Application No. 202411260013.2 filed on September 9, 2024, the disclosure of which is hereby incorporated by reference in its entirety.

### BACKGROUND

Ultrasound is the most commonly used and reliable first-line method for evaluating lesions in clinical practice, which can assist doctors in achieving accurate diagnoses. Currently, the common examination method involves performing a sector scan of lesions in two-dimensional (2D) mode and issuing corresponding textual descriptions and diagnostic conclusions in ultrasound reports for clinical reference. However, existing ultrasound reports present comprehension challenges for clinicians, who can only rely on their experience and spatial reasoning. This typically necessitates further consultation with sonographers, which is time-consuming and laborious. Uterine lesions are explained below as an example.

The uterus is one of the most critical organs in the female reproductive system, playing a vital role in sustaining hormone secretion and fertility. Anatomically, it comprises the cervix and uterine corpus, the latter consisting mainly of the endometrium, myometrium, and supporting tissues. Attributable to evolving living environments and lifestyles associated with industrialized societies, the prevalence of various uterine lesions has risen gradually, garnering significant clinical concern. For instance, uterine fibroids (benign tumors representing the most prevalent gynecological neoplasm) affect 70-80% of women during their reproductive years, with peak incidence occurring between ages 30 and 50. These fibroids may present with symptoms including abnormal bleeding, pelvic pain, and infertility, contributing to substantial morbidity impacting both physical and psychosocial well-being. Consequently, achieving both timely detection and accurate diagnosis of uterine lesions is critically important.

Clinically, precise localization of uterine lesions (including their intrauterine position, quantity, and dimensions), combined with typing, grading, and longitudinal change assessment is essential for developing appropriate treatment strategies. Taking uterine fibroids as an example, patients typically exhibit strong fertility preservation requirements while desiring uterine retain for both physiological and psychosocial considerations. Consequently, early clinical intervention is warranted to mitigate potential malignant transformation risks. Treatment plans are formulate primarily based on fibroids distribution characteristics (location, type, number, and size) alongside patient preferences. Thus, rapid and accurate acquisition of fibroid distribution data is critically important for clinical decision-making.

According to established guidelines and clinical practice, ultrasound remains the preferred first-line modality for evaluating uterine fibroids, offering high diagnostic sensitivity and specificity. Its ability to accurately characterize fibroid distribution and typing confers significant clinical utility. Comprehensive preoperative mapping of fibroid distribution enables optimal surgical planning, minimizes intraoperative trauma and postoperative morbidity, shortens recovery duration, and maximizes fertility preservation potential. Furthermore, it facilitates longitudinal monitoring of disease progression. Currently, however, ultrasound findings are communicated solely via textual descriptions in reports, creating significant interpretation barriers for clinicians. Gynecologists primarily rely on spatial reasoning supplemented by verbal consultations with sonographers to comprehend fibroid distribution, a cumbersome and inefficient workflow that impedes evidence-based clinical decision-making.

Therefore, interdepartmental communication efficiency regarding lesion distribution data between sonologists and clinicians requires refinement.

### SUMMARY

The present disclosure relates to medical devices, specifically to ultrasound imaging apparatus and methods for presenting lesion distribution.

The present disclosure mainly provides ultrasound imaging apparatus and methods for presenting lesion distribution, designed to enhance inter-clinician communication efficiency regarding patient-specific lesion distribution data.

An ultrasound imaging apparatus provided in some embodiments may include:

an ultrasound probe, configured to emit ultrasound waves and receive corresponding ultrasound echoes;

a transmit and receive control circuit, configured to control the ultrasound probe to emit the ultrasound waves and receive the ultrasound echoes; and

a processor, configured to:

obtain volumetric data containing target tissue and target lesion;

identify the target tissue from the volumetric data, and generate the three-dimensional (3D) contour of the target tissue;

identify the target lesion from the volumetric data, and generate the 3D contour of the target lesion;

generate a 3D schematic diagram based on the 3D contour of the target tissue and the 3D contour of the target lesion, wherein the 3D schematic diagram represents the morphology of the target tissue and the morphology and location of the target lesion in the target tissue; and

generate a 2D schematic diagram corresponding to at least one target section based on the 3D contour of the target tissue and the 3D contour of the target lesion; wherein the 2D schematic diagram corresponding to the target section comprises: a target tissue graphic representing the morphology of the target tissue, and a target lesion graphic representing the morphology and location of the target lesion; and the target section comprises an anatomical plane of the target tissue.

A method for presenting lesion distribution provided in some embodiments may include:

obtaining volumetric data containing target tissue and target lesion;

identifying the target tissue from the volumetric data, and generating the 3D contour of the target tissue;

identifying the target lesion from the volumetric data, and generating the 3D contour of the target lesion;

generating a 3D schematic diagram based on the 3D contour of the target tissue and the 3D contour of the target lesion, wherein the 3D schematic diagram represents the morphology of the target tissue, and the morphology and location of the target lesion in the target tissue; and

generating a 2D schematic diagram corresponding to at least one target section based on the 3D contour of the target tissue and the 3D contour of the target lesion, wherein the 2D schematic diagram corresponding to the target section comprises a target tissue graphic representing the morphology of the target tissue and a target lesion graphic representing the morphology and location of the target lesion, and the target section comprises an anatomical plane of the target tissue.

A computer-readable storage medium is provided in some embodiments, wherein the medium stores a program being executable by a processor to implement the method as described above.

Based on the ultrasound imaging apparatus and the method for presenting lesion distribution described in the foregoing embodiments, volumetric data is obtained. The target tissue and target lesion are subsequently identified from the volumetric data to obtain their 3D contours. Utilizing these dual contours, a 3D schematic diagram is generated to represent the morphology of the target tissue and the morphology and location of the target lesion within the target tissue. This approach facilitates intuitive spatial comprehension of lesion distribution, surpassing conventional ultrasound images in interpretability, thereby enhancing clinical workflow efficiency. Additionally, a 2D schematic diagram corresponding to at least one target section can be generated based on the 3D contours of the target tissue and lesion. The 2D schematic diagram corresponding to the target section includes: a target tissue graphic representing the morphology of the target tissue, and a target lesion graphic representing the morphology and location of the target lesion. In this way, users can also understand the distribution of lesions through the 2D schematic diagram; and clinicians can convey the distribution of lesions through both the 3D and 2D schematic diagrams without relying on specialist-dependent sonographic image interpretation.

### BRIEF DESCRIPTION OF THE DRAWINGS

FIG. 1 is a block diagram of a terminal device provided in some embodiments of the present disclosure;

FIG. 2 is a flowchart of a method for presenting lesion distribution provided in some embodiments of the present disclosure;

FIG. 3 is a block diagram of an ultrasound imaging apparatus provided in some embodiments of the present disclosure;

FIG. 4 is a schematic diagram of 2D ultrasound images of ultrasound volumetric data and its sagittal, transverse and coronal planes;

FIG. 5 is a schematic diagram of a 3D contour and a 2D contour of a target tissue;

FIG. 6 is a schematic diagram of a 3D schematic diagram and 2D schematic diagrams corresponding to three target sections displayed on a display interface;

FIG. 7 is a 2D schematic diagram corresponding to a target section in some embodiments;

FIG. 8 is a schematic diagram showing the overlap of a target lesion graphic and 2D contours of major anatomical structures;

FIG. 9 is a 2D schematic diagram corresponding to a target section in some embodiments;

FIG. 10 is a tumor classification reference atlas in some embodiments of the present disclosure; and

FIG. 11 is a schematic diagram of a 3D schematic diagram and at least two mutually perpendicular 2D ultrasound images displayed on a display interface.

### DETAILED DESCRIPTION

Specific embodiments of the present disclosure will now be described in detail with reference to the accompanying drawings. Similar or related components in different embodiments are labeled with associated reference numerals. The following embodiments include detailed descriptions to facilitate understanding of the present disclosure. However, those skilled in the art will readily recognize that certain features may be omitted under specific circumstances or substituted by other components, materials, or methods. In some instances, certain operations related to the present disclosure are not explicitly described or illustrated herein. This intentional exclusionis intentional to avoid obscuring the core technical solutions of the present disclosure. For those skilled in the art, a complete understanding of these operations can be attained through the descriptions provided in this specification and general technical knowledge in the art.

Additionally, the features, operations, or characteristics described in the specification may be combined in any suitable manner to form various embodiments. Similarly, steps or actions in the method descriptions may be reordered or modified in ways that would be obvious to those skilled in the art. Therefore, the sequences presented in the specification and drawings are intended solely to clarify the description of specific embodiments and do not imply mandatory orderings, unless explicitly stated that a particular sequence is required.

The numerical designations assigned to components in this specification, such as 'first,' 'second,' or similar ordinal terms, serve solely to distinguish described objects and carry no inherent sequential or technical implications. Furthermore, the terms 'connected' and 'coupled' as used herein encompass both direct and indirect connection (coupling), unless explicitly stated otherwise.

In view of the clinical background and existing problems mentioned in the background technology, the present disclosure can, after a doctor acquires 3D volumetric data containing tissues and lesions, the system automatically or semi-automatically process the 3D volumetric data to obtain the 3D contour of the tissue and automatically or semi-automatically process the 3D volumetric data to obtain the 3D contour of the lesion. Thus, the tissue and lesion can be presented 3Dly and stereoscopically based on these two 3D contours. Moreover, a 2D lesion distribution schematic diagram containing the graphics of the tissue and lesion can be obtained based on the 3D contours of the tissue and lesion. Through these 3D and 2D schematic diagrams, the distribution of lesions can be understood more intuitively and accurately in clinical practice, providing a basis for decision-making in formulating subsequent treatment plans. The following provides detailed explanations through some embodiments.

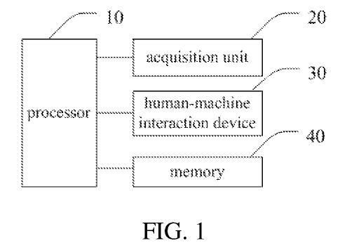

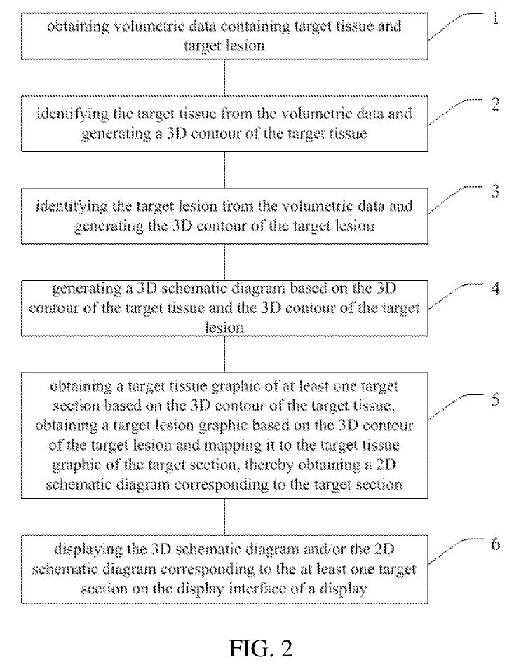

Some embodiments of the present disclosure provide a terminal device that can automatically or semi-automatically generate and display 3D and 2D schematic diagrams for presenting the location and distribution of lesions, as shown in FIG. 1. It may include a processor 10 and an acquisition unit 20. The generation of 3D and 2D schematic diagrams by the terminal device may be shown in FIG. 2, including the following steps:

Step 1: obtaining volumetric data containing target tissue and target lesion. For example, the processor 10 acquires volumetric data containing the target tissue and the target lesion via the acquisition unit 20. The volumetric data is 3D data containing the target tissue and the target lesion. Clinicians usually need to accurately understand the location of the lesion in the tissue for subsequent examinations and surgeries, etc. In this embodiment, the volumetric data is 3D volumetric data, which may be obtained by scanning the target tissue with an ultrasound imaging device, a computed tomography (CT) device, a digital X-ray photography system (DR), or a magnetic resonance imaging (MRI) device. This embodiment takes the volumetric data as ultrasound volumetric data as an example for illustration.

The volumetric data may be obtained from an external device, for example, an external device stores the volumetric data. The acquisition unit 20 may be a communication module that acquires the volumetric data from the external device through wired or wireless means.

The volumetric data may also be generated by the terminal device itself. For example, the processor 10 acquires ultrasound volumetric data containing the target tissue and the lesion by performing 3D ultrasound scanning on the target tissue of a patient via the acquisition unit 20. This embodiment takes this as an example for illustration, that is, in this embodiment, the terminal device is an ultrasound imaging apparatus.

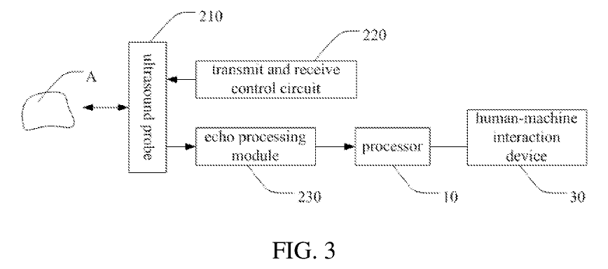

As shown in FIG. 3, the acquisition unit 20 of the ultrasound imaging apparatus includes an ultrasound probe 210, a transmit and receive control circuit 220, and an echo processing module 230.

The ultrasound probe 210 is configured to transmit ultrasound waves to the target tissue A (a region of interest) and receive corresponding ultrasound echo signals to obtain ultrasound data, such as 2D or 3D ultrasound data. In some specific embodiments, the ultrasound probe 210 includes multiple transducer elements, which are configured to mutually convert electrical pulse signals and ultrasound waves, thereby enabling the transmission of ultrasound waves to the target tissue A and the reception of the corresponding ultrasound echo signals. The transducer elements can emit ultrasound waves according to the excitation of electrical signals or convert the received ultrasound waves into electrical signals. Therefore, each transducer element can be used to transmit ultrasound waves to the target tissue A or receive the ultrasound echo waves returned from the tissue. During ultrasound detection, the transducer elements used for transmitting ultrasound waves and those used for receiving ultrasound echo signals can be controlled by a transmission sequence and a reception sequence. All transducer elements involved in ultrasound wave transmission can be simultaneously excited by electrical signals to simultaneously transmit ultrasound waves; or the transducer elements involved in ultrasound wave transmission can also be excited by several electrical signals with a certain time interval, thereby continuously transmitting ultrasound waves with a certain time interval.

The transmit and receive control circuit 220 is configured to control the ultrasound probe 210 to perform the transmission of ultrasound waves and the reception of ultrasound echo signals. For example, the transmit and receive control circuit 220 is used on the one hand to control the ultrasound probe 210 to transmit ultrasound waves to the target tissue A, and on the other hand to control the ultrasound probe 210 to receive the ultrasound echo signals reflected by the region of interest. In some specific embodiments, the transmit and receive control circuit 220 is used to generate the transmission sequence and the reception sequence and output them to the ultrasound probe 210. The transmission sequence is configured to control some or all of the multiple transducer elements in the ultrasound probe 210 to transmit ultrasound waves to the target tissue A. The parameters of the transmission sequence may include the number of transducer elements used for transmission, and the transmission parameters for the ultrasound waves (such as amplitude, frequency domain, number of transmissions, transmission interval, transmission angle, wave type, and/or focusing location, etc.). The reception sequence is configured to control some or all of the multiple transducer elements to receive the echoes after the ultrasound waves pass through the tissue. The parameters of the reception sequence may include the number of transducer elements used for reception and the reception parameters of the echoes (such as reception angle, depth, etc.). Depending on the different uses of the ultrasound echoes or the different images generated based on the ultrasound echoes, the ultrasound wave parameters in the transmission sequence and the echo parameters in the reception sequence may also be different.

The echo processing module 230 is configured to process the ultrasound echo signals received by the ultrasound probe 210, such as filtering, amplifying, and beamforming the ultrasound echo signals to obtain ultrasound image data. In specific embodiments, the echo processing module 230 may output the ultrasound image data to the processor 10, or first store the ultrasound image data in a memory 40. When operations based on the ultrasound image data are needed, the processor 10 reads the ultrasound image data from the memory. Those skilled in the art should understand that in some embodiments, when there is no need to filter, amplify, or beamform the ultrasound echo signals, the echo processing module 230 may also be omitted. In some embodiments, some or all of the functions of the echo processing module 230 may also be performed by the processor 10, that is, the echo processing module 230 can be a part of the processor 10.

The processor 10 is configured to obtain the ultrasound image data and use relevant algorithms to obtain the required parameters or images. In some embodiments of the present disclosure, the processor 10 includes, but is not limited to, a central processing unit (CPU), a microcontroller unit (MCU), a field-programmable gate array (FPGA), and a digital signal processor (DSP), and other devices used for interpreting computer instructions and processing data in computer software. In some embodiments, the processor 10 is configured to execute various computer applications in the computer-readable storage medium, thereby realizing the various functions of the ultrasound imaging device. In this embodiment, the ultrasound volumetric data is beamformed 3D ultrasound image data. For example, it may be ultrasound image data that has not been processed and cannot be directly displayed on the display, or it may be ultrasound images that have been processed and can be directly displayed on the display.

The ultrasound imaging apparatus may also include a human-machine interaction device 30. The human-machine interaction device 30 is used for human-machine interaction, that is, it is configured to receive user input and output visual information. It can receive user input via a keyboard, an operation button, a mouse, a trackball, etc., or via a touch screen integrated with the display; and it can output visual information via a display. The display may be configured to show information, such as parameters and images calculated by the processor 10. It should be understood by those skilled in the art that in some embodiments, the ultrasound imaging apparatus itself may not integrate a display but instead connect to an external display device to show information through the external display device.

It should be noted that the structure shown in FIG. 3 is merely illustrative and may include more or fewer components than those shown in FIG. 3, or have a different configuration from that shown in FIG. 3. The components shown in FIG. 3 can be implemented in hardware and/or software.

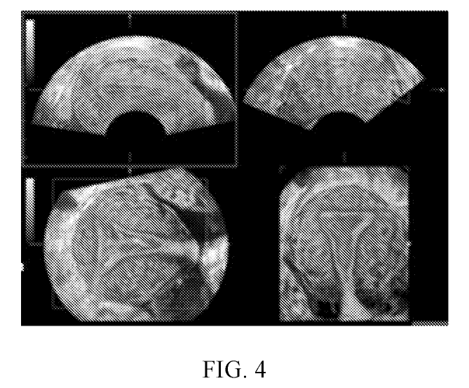

The target tissue may be various tissues of the organism, such as the uterus, heart, thyroid, ovary, kidney, liver, etc. This embodiment takes the uterus as an example to illustrate. During the process of examining a patient's uterus, the patient is placed in a lithotomy position and a routine transvaginal 3D ultrasound examination is performed to determine the location and basic conditions of the uterus. The scanning angle and position of the vaginal volume probe are adjusted to ensure that all major anatomical structures of the uterus (such as the endometrium, uterine body, and cervix, etc.) are within the scanning range. The processor 10 controls the ultrasound probe 210 to emit ultrasound waves to the target tissue (such as the uterus) through the transmit and receive control circuit 220, and receive the ultrasound echoes reflected back from the target tissue and convert them back into electrical signals. The echo processing module 230 performs signal processing such as beam forming on the electrical signals, and the processor 10 performs 3D reconstruction on the signals. After post-processing such as image rendering, the ultrasound volumetric data is obtained. The processor 10 can also output the ultrasound volumetric data to the display of the human-machine interaction device 30 for visualization display (as shown in the 3D diagram in the lower right corner of FIG. 4), and can also display the 2D ultrasound images of the sagittal plane, transverse plane, and coronal plane of the ultrasound volumetric data on the display, as shown in the three 2D diagrams in FIG. 4, which is convenient for doctors to view the location of the lesion in the target tissue. The above process may be the steps of the ultrasound imaging apparatus performing a routine 3D ultrasound scan on the target tissue and its lesion, without the need for additional ultrasound scans.

Step 2: identifying the target tissue from the volumetric data and generating a 3D contour of the target tissue by the processor 10. This process may be automatically completed by the processor 10 or semi-automatically with the assistance of a user. There are various ways to do this, and several are described below for illustration.

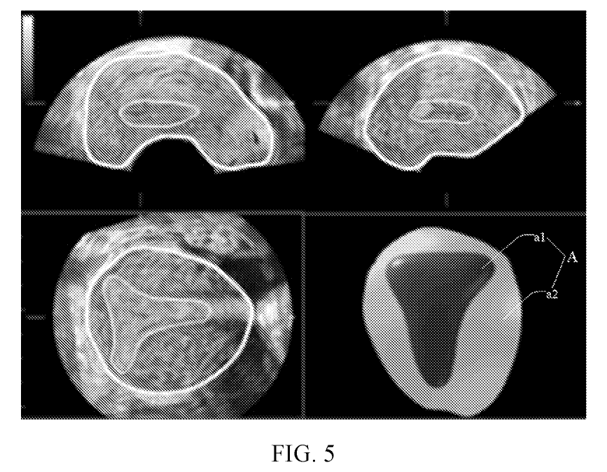

In one way, step 2 is automatically completed by the processor 10, which directly identifies the target tissue from the ultrasound volumetric data. The processor 10 automatically extracts a plurality of major anatomical structures of the target tissue from the volumetric data, and then generates a 3D contour A of the target tissue based on the plurality of major anatomical structures of the target tissue, as shown in the 3D diagram in the lower right corner of FIG. 5. In the case of uterine fibroids, the major anatomical structures of the uterus may include multiple types such as the endometrium, uterine body, and cervix. In this embodiment, the major anatomical structures of the uterus include the endometrium and the uterine body.

The processor 10 may automatically extract the plurality of major anatomical structures of the target tissue from the volumetric data. There are many methods for extraction. For example, traditional gray-scale and/or morphological feature detection methods may be used to detect the endometrium and uterine body; alternatively, machine learning or deep learning methods may be used to detect or precisely segment the corresponding anatomical structures in the volumetric data. The following is a detailed description.

The processor 10 may automatically segment a plurality of major anatomical structures of the target tissue from volumetric data based on traditional methods. Whether in 2D or 3D ultrasound images, the endometrium and uterine body have distinct echo difference boundaries from surrounding tissues. Target detection methods (such as point detection, line detection) may be used to detect the regions where the target tissues are located (such as the endometrium and uterine body), and then these regions may be segmented to obtain the plurality of major anatomical structures of the target tissue. Commonly used segmentation algorithms include level set (Level Set) segmentation algorithm, random walk (Random Walk), graph cut (Graph Cut), Snake, etc. When the ultrasound volumetric data is formed by fusing multiple 2D ultrasound images scanned by the probe, the above segmentation algorithms are applied to these 2D ultrasound images, and then the segmentation results are fused to obtain the final 3D segmentation result, that is, to segment multiple major anatomical structures of the target tissue. If the ultrasound volumetric data is directly scanned by the probe, the above segmentation algorithms are directly applied to the volumetric data to obtain the 3D segmentation result, that is, to segment multiple major anatomical structures of the target tissue.

The processor 10 may also achieve the detection of the major anatomical structures based on machine learning or deep learning methods, that is, automatically segment multiple major anatomical structures of the target tissue from volumetric data based on machine learning or deep learning methods. Currently, another common method for target region detection and segmentation mainly involves applying machine learning or deep learning algorithms to the above-mentioned ultrasound image data (volumetric data), allowing the algorithm to learn and build a model to automatically detect or precisely segment multiple major anatomical structures of the target tissue. This method mainly includes: constructing an expert database, the algorithm learning the features or patterns of the target regions in the database, and the algorithm automatically detecting and segmenting the major anatomical structures in other data based on the learned features or patterns. Specifically in this disclosure, an expert database is pre-constructed, where the expert database refers to the marking of multiple major anatomical structures of the target tissue in the above-mentioned ultrasound image data based on the knowledge and experience of experts. Taking the detection and segmentation of the endometrium as an example, in this disclosure, the endometrium region may be manually marked first as the target region. The processor 10, based on the existing expert database marking, applies machine learning and/or deep learning algorithms to learn the expert database, obtaining the features or patterns that distinguish the endometrium from other regions to achieve the automatic detection and segmentation of the endometrium. The detection and segmentation of the uterine body are similar, and the endometrium is just used as an example for illustration. After the machine learning and/or deep learning algorithms learn the expert database, the specific implementation of the automatic detection and segmentation of multiple major anatomical structures in the volumetric data includes but is not limited to the following methods.

A first method is to adopt the traditional feature extraction combined with the discriminator classification approach. For instance, a common method in traditional approaches is a sliding window-based approach, that is: firstly, the processor 10 performs feature extraction on a region within a sliding window, where the feature extraction methods may be traditional ones such as PCA, LDA, Haar features, texture features, etc., or deep neural networks may be used for feature extraction; then, the extracted features are matched with the expert database, and discriminators such as KNN, SVM, random forest, and neural networks are used for classification to determine whether the current sliding window is a region of interest (a region where the major anatomical structures are located), and if it is, a category corresponding thereto is obtained (determining the category of the major anatomical structure), thus automatically segmenting the various major anatomical structures of the target tissue from the volumetric data.

A second method is to use the deep learning-based Bounding-Box detection and recognition approach. A common way is in that: the processor 10 learns the features and regresses the parameters of the constructed expert database by stacking the basic convolutional layers and fully connected layers. For the input volumetric data, the corresponding Bounding-Box of the region of interest (the region where the major anatomical structures are located) can be directly regressed through the network, and the category of the tissue structure within the region of interest is also obtained (determining the category of the major anatomical structure). Common networks include R-CNN, Fast R-CNN, Faster-RCNN, SSD, YOLO, etc. Thus, the various major anatomical structures of the target tissue are automatically segmented from the volumetric data.

A third method is an end-to-end semantic segmentation network approach based on deep learning. This type of method is similar in structure to the second method based on deep learning Bounding-Box, but the difference lies in removing the fully connected layers and adding upsampling or deconvolution layers to make the input and output dimensions the same, thereby directly obtaining the region of interest and its corresponding category of the input volumetric data. Common networks include FCN, U-Net, Mask R-CNN, etc.

A fourth method is to only use the first, second, or third method to locate the major anatomical structures, and then design an additional classifier based on the resulted locations to classify and determine the major anatomical structures, that is, to determine the category of each major anatomical structure. A common classification and determination method may involve: performing feature extraction firstly on the region of interest (ROI) or a mask, where the feature extraction methods may be traditional ones such as PCA, LDA, Haar features, texture features, etc., or deep neural networks; then, matching the extracted features with the expert database, and using discriminators such as KNN, SVM, random forest, and neural networks for classification, thereby automatically segmenting the various major anatomical structures of the target tissue from the volumetric data.

Similarly, the machine learning and deep learning algorithms mentioned above are currently mature target segmentation algorithms. The present disclosure uses machine learning or deep learning algorithms to precisely segment the major anatomical structures of the endometrium and other uterine structures. Using other algorithms to achieve the purpose of segmenting the various major anatomical structures from the volumetric data is also acceptable, and the present disclosure does not impose any restrictions.

In another approach, step 2 is automatically or semi-automatically completed by the processor 10. This approach requires first slicing the ultrasound volumetric data to obtain multiple 2D ultrasound images. After identifying the target tissue from the 2D ultrasound images, a 3D contour is generated based on the contours of the target tissue in each 2D ultrasound image. The processor 10 then segments multiple 2D cross-sectional ultrasound images from the volumetric data. For instance, the processor 10 can slice the volumetric data by continuously or at certain intervals sampling in three perpendicular 2D planes to obtain multiple 2D cross-sectional ultrasound images. Specifically, the processor 10 establishes a spatial coordinate system and slices (samples) the volumetric data with multiple 2D planes parallel to the XOY plane to obtain multiple 2D cross-sectional ultrasound images; slices (samples) the volumetric data with multiple 2D planes parallel to the XOZ plane to obtain multiple 2D cross-sectional ultrasound images; and slices (samples) the volumetric data with multiple 2D planes parallel to the YOZ plane to obtain multiple 2D cross-sectional ultrasound images. The processor 10 can also slice the volumetric data by continuously or at certain angular intervals sampling from a certain location, such as the center of the volumetric data, to obtain multiple 2D cross-sectional ultrasound images.

Then, the processor 10 can automatically identify the 2D contours of multiple major anatomical structures of the target tissue from these 2D cross-sectional ultrasound images. FIG. 5 shows the 2D contours of the endometrium and uterine body on the sagittal, transverse, and coronal planes of the uterus (indicated by the white lines in the figure). There are many methods for automatic identification. For example, traditional gray-scale and/or morphological feature detection methods may be used to detect major anatomical structures (such as the endometrium and uterine body); machine learning or deep learning methods can also be used to detect or precisely segment the corresponding major anatomical structures in these 2D cross-sectional ultrasound images. The following is a detailed introduction.

The major anatomical structures may be detected (recognized) based on traditional methods, which may be described as follows. In the above-mentioned 2D cross-sectional ultrasound images, the endometrium and the uterine body have distinct echo difference boundaries from the surrounding tissues. Traditional feature detection methods such as morphology may be used to detect the contours of the endometrium and the uterine body. For example, the processor 10 first performs binary segmentation on the above-mentioned 2D cross-sectional ultrasound images, and after corresponding morphological operations, multiple possible regions are obtained. Then, for each possible region, the probability that the region is a major anatomical structure is determined based on features such as shape and gray-scale brightness. The region with the highest probability is taken as the region of the major anatomical structure. In the detected region of the major anatomical structure, the contour of the major anatomical structure is found based on morphological features, thereby completing the recognition or detection of the 2D contour of the major anatomical structure. Of course, other traditional gray-scale detection and segmentation methods may also be used, such as Otsu thresholding, level set, graph cut, and Snake.

The major anatomical structures may be detected (recognized) based on machine learning or deep learning methods, which may be described as follows. Currently, another common method for target region detection and segmentation mainly involves applying machine learning or deep learning algorithms to the above-mentioned 2D cross-sectional ultrasound images, allowing the algorithm to learn and construct a model to automatically detect or precisely segment the major anatomical structures in the 2D cross-sectional ultrasound images. This method mainly includes: constructing an expert database, the model learning the features or patterns of the target regions in the expert database, and the model automatically detecting and segmenting the major anatomical structures in the 2D cross-sectional ultrasound images based on the learned features or patterns. Specifically in this disclosure, an expert database is pre-constructed. The expert database refers to a database formed by marking the various major anatomical structures in the 2D ultrasound images of various target tissues based on the knowledge and experience of experts. Taking the detection and segmentation of the endometrium as an example, in this disclosure, the endometrium region may be first manually marked as the target region. The processor 10, based on the existing expert database marking, applies machine learning and/or deep learning algorithms to learn the expert database, obtaining the features or patterns that distinguish the endometrium from other regions, to achieve the automatic detection and segmentation of the endometrium. The detection and segmentation of the uterine body are the same. After the machine learning and/or deep learning algorithms learn the expert database, the specific implementation of the automatic detection and segmentation of multiple major anatomical structures in the 2D cross-sectional ultrasound images can adopt the first to fourth methods mentioned above, only changing the volumetric data to 2D cross-sectional ultrasound images, which will not be repeated here.

Of course, the processor 10 can also obtain the 2D contours of multiple major anatomical structures of the target tissue in these 2D cross-sectional ultrasound images based on the drawing operations performed by the user on these 2D cross-sectional ultrasound images. For example, the processor 10 displays these 2D cross-sectional ultrasound images on the display interface of the display, and at the same time displays a drawing toolbar to provide corresponding drawing functions. The drawing toolbar can allow users to draw on the 2D cross-sectional ultrasound images, thereby drawing the 2D contours of the major anatomical structures.

Whether the processor 10 automatically or with the help of a user's drawing operations, the processor 10 can fit (reconstruct) the 3D contour of the target tissue based on the 2D contours of multiple major anatomical structures in these 2D cross-sectional ultrasound images. Specifically, the 3D contour of the target tissue may be fitted by methods such as the least squares method or nonlinear fitting method. The 3D contour of the target tissue includes the 3D contours of each major anatomical structure. As shown in the lower right corner of FIG. 5, the 3D contour of the uterus is presented, where the red region is the 3D contour a1 of the endometrium, and the white or semi-transparent region surrounding the endometrium is the 3D contour a2 of the uterine body.

Step 3: identifying the target lesion from the volumetric data and generating the 3D contour of the target lesion by the processor 10. The specific process may be the same as that in Step 2, only replacing the target tissue with the target lesion. Similarly, this process may be automatically completed by the processor 10 or semi-automatically completed with the assistance of the user. There are various specific methods, and several are listed below for illustration.

In one way, step 3 is automatically completed by the processor 10. This method directly identifies the target lesion from the volumetric data and automatically segments the 3D contour of the target lesion. For example, the processor 10 precisely segments the target lesion such as uterine fibroids through machine learning or deep learning algorithms. Of course, other algorithms that can achieve the purpose of image segmentation can also be used. The specific process of this method can be the same as the automatic and direct identification of the target tissue in Step 2, only changing the target tissue and its major anatomical structures to the target lesion. This will not be elaborated here.

In another way, Step 3 may be semi-automatically completed by the processor 10. The target lesion in the volumetric data may be found by users manually, and then be segmented automatically by the processor 10. For example, the processor 10 displays the volumetric data on the display interface. The user can manually perform operations such as translation and rotation on the displayed volumetric data to find the uterine fibroids existing in the volumetric data. That is, for example, the processor 10 receives a user's input translation instructions to translate the volumetric data, and/or receives the user's input rotation instructions to rotate the volumetric data. After the user finds the target lesion (such as uterine fibroids), they can mark the target lesion, such as clicking or drawing lines within the region where the uterine fibroids are located to indicate the location of the uterine fibroids. In this way, the processor 10 can perform segmentation and recognition based on the location indication of the marking. The processor 10 obtains the location of the target lesion marked by a user based on the user's marking operation on the target lesion in the volumetric data; and then automatically segments the 3D contour of the target lesion based on the location of the target lesion marked by the user. The specific process of the processor 10 automatically segmenting the 3D contour of the target lesion may be the same as the automatic and direct identification of the target tissue in Step 2, only changing the target tissue and its major anatomical structures to the target lesion. That is, after the user marks the target lesion, the subsequent specific process of automatic segmentation may be the same as the content described in the previous paragraph, and will not be repeated here.

In yet another way, the processor 10 obtains the size of the target lesion based on a user measurement operation on the target lesion in the volumetric data; and fits to obtain the 3D contour of the target lesion based on the size of the target lesion. For example, the processor 10 displays the volumetric data on the display interface of a display. Operations such as translation and rotation can be performed manually by a user on the displayed volumetric data to find the uterine fibroids existing in the volumetric data and the three diameters (length, width, and thickness) of the fibroids, or any two of the three diameters, can be measured. The processor 10 automatically generates the 3D contour of the fibroids in the 3D space based on the measured values (at least two of the length, width, and thickness) and the location of the measurement points. If three diameters are measured, the processor 10 automatically generates an ellipsoid contour with the same three diameters as the 3D contour of the target lesion; if only two of the three diameters are measured, the processor 10 can take one of the measured diameters as the third diameter and automatically fit to generate an ellipsoid contour with the corresponding diameters as the 3D contour of the target lesion.

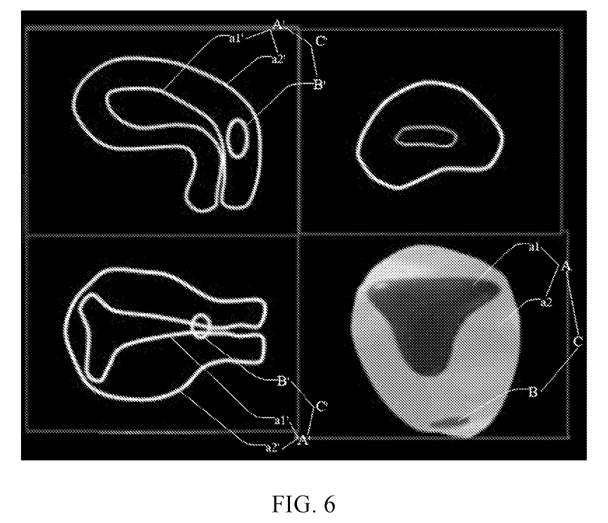

Step 4: generating a 3D schematic diagram C by the processor 10 based on the 3D contour of the target tissue and the 3D contour of the target lesion, as shown in the 3D diagram in the lower right corner of FIG. 6. The 3D schematic diagram is used to represent the morphology of the target tissue and the morphology and location of the target lesion within the target tissue. That is, after the 3D schematic diagram is displayed on the monitor, clinicians, patients, and their families can very intuitively see the morphology of the target tissue and also the morphology and location of the target lesion within the target tissue.

Specifically, the processor 10 can obtain the location of the target lesion within the target tissue, and then fuse the 3D contour of the target lesion into the 3D contour of the target tissue based on the location of the target lesion within the target tissue to obtain the 3D schematic diagram C. For example, the contour or image at the location of the target lesion in the 3D contour of the target tissue can be replaced with the 3D contour of the target lesion, thereby obtaining the 3D schematic diagram C corresponding to the target tissue and the target lesion in the volumetric data.

There may be various methods for the processor 10 to obtain the location of the target lesion in the target tissue. The following describes one method for illustration. Regardless of the specific way or method (see the above step 2) used by the processor 10 to identify the target tissue from the volumetric data, it actually knows (obtains) the spatial locations of each major anatomical structure relative to the volumetric data, that is, it obtains the spatial locations of each major anatomical structure in the volumetric data. The processor 10 can take the volumetric data as a reference (such as the spatial coordinate system established based on the volumetric data as a reference), and based on the spatial locations of each major anatomical structure in the volumetric data, obtain the spatial positional relationships among each major anatomical structure. That is, the processor 10 can obtain the spatial positional relationships among each major anatomical structure during the execution of step 2. Similarly, regardless of the specific way or method (see the above step 3) used by the processor 10 to identify the target lesion from the volumetric data, it actually knows (obtains) the spatial location of the target lesion relative to the volumetric data, that is, it obtains the spatial location of the target lesion in the volumetric data. The processor 10 can take the volumetric data as a reference (such as the spatial coordinate system established based on the volumetric data as a reference), and based on the spatial location of the target lesion in the volumetric data and the spatial location of the target tissue in the volumetric data, obtain the location of the target lesion in the target tissue. Specifically, the processor 10 can obtain the spatial positional relationships between the target lesion and each major anatomical structure based on the spatial location of the target lesion in the volumetric data and the spatial locations of each major anatomical structure in the volumetric data.

In some embodiments, the processor 10 can also automatically generate the pedicle of the lesion. For example, the processor 10 generates the 3D contour of the pedicle of the lesion in the 3D schematic diagram based on the location of the target lesion in the target tissue. The 3D contour of the target tissue is connected to the 3D contour of the target lesion through the 3D contour of the lesion pedicle. The 3D contour of the target tissue in the 3D schematic diagram includes the 3D contours of multiple major anatomical structures of the target tissue. The point, on the 3D contour of the major anatomical structure adjacent to the 3D contour of the target lesion, that is closest to the 3D contour of the target lesion is the 3D attachment point of the lesion pedicle. That is, if the 3D contour of the target lesion is connected to the 3D contour of the adjacent major anatomical structure with the shortest line segment, the attachment point on the 3D contour of the major anatomical structure is the 3D attachment point of the lesion pedicle.

In some embodiments, the appearance of the 3D contour of the target lesion in the 3D schematic diagram can reflect the benign or malignant nature of the lesion, and even the degree of malignancy. Benign, malignant, and degree of malignancy may all be referred to as the type of the lesion. The processor 10 can identify the type of the target lesion in the volumetric data, and the specific methods may be the same as those mentioned above for identifying the target lesion and target tissue, and will not be repeated here. Of course, there are only so many types of lesions, and users may manually input the type of the lesion. For example, the processor 10 can display multiple types on the display interface of the display for users to select. The type of the target lesion may at least be divided into benign and malignant. In this embodiment, malignant is further divided into degrees of malignancy, that is, the type of the target lesion may be classified into benign and multiple degrees of malignancy, which may be selected in the system by a user through a knob or button, etc. The processor 10 receives the type selected by the user and determines the type selected by the user as the type of the target lesion in the volumetric data. One type may correspond to a specific appearance of the 3D contour of the target lesion in advance. The appearance of the 3D contour may refer to the outer surface of the 3D contour. For example, benign corresponds to a smooth outer surface, and malignant corresponds to a rough outer surface. The higher the degree of malignancy of the type, the rougher the outer surface. In the 3D schematic diagram of the uterine scene, the 3D contour of a benign fibroid is a smooth ellipsoid, and the 3D contour of a malignant fibroid is an ellipsoid with spicules. The processor 10 then determines the appearance of the 3D contour of the target lesion based on the type of the target lesion. With the displayed 3D schematic diagram in subsequent, the user can know the type of the lesion by observing the outer surface of the 3D contour of the target lesion, which is very intuitive. Of course, in addition to the roughness of the outer surface, other distinctive appearance designs can also be used to achieve the purpose of distinguishing the type of lesion, and this is not limited here.

To better reflect the spatial relationship differences among various major anatomical structures and target lesions, in the 3D schematic diagram, the 3D contours of each major anatomical structure and the 3D contours of the target lesions can be distinguished by different markers. The markers may be characters (such as text, letter codes, numbers, etc.) or colors, as long as they can be distinguished. In this embodiment, as shown in FIG. 6, different colors are used for distinction.

The target lesion and a few major anatomical structures may be located inside other major anatomical structures. In such cases, the 3D schematic diagram of the outer major anatomical structure may obscure the target lesion and the major anatomical structure inside. To better present the internal structure and lesion of the tissue, the processor 10 can determine whether one major anatomical structure is located inside another major anatomical structure based on the spatial (positional) relationship between the major anatomical structures. If so, the 3D contour of the other major anatomical structure in the 3D schematic diagram is set to be transparent or semi-transparent, that is, the 3D contour of the outer major anatomical structure is set to be transparent or semi-transparent, so that it allows users to see the inner major anatomical structure. Similarly, the processor 10 can also determine whether the target lesion is located inside a major anatomical structure based on the spatial relationship between the major anatomical structures and the target lesion. If so, the 3D contour of the major anatomical structure in the 3D schematic diagram is set to be transparent or semi-transparent, that is, the 3D contour of the major anatomical structure outside the target lesion is set to be transparent or semi-transparent, so that the users can see the inner target lesion.

In some embodiments, the 3D diagram C may be rotated at a certain angle, orientation and rate by a user configuration, and the rotating video may be exported for offline viewing by the user later. For example, the human-machine interaction device 30 may be provided with a function key (which may be a physical key or a virtual key). When the function key is triggered by the user, the processor 10 displays a setting interface on the display interface of the human-machine interaction device 30. The setting interface has the 3D diagram C and provides rotation parameters of the 3D diagram C for users to set. The rotation parameters may be at least one of angle, orientation and rate. The processor 10 can rotate the 3D diagram C on the setting interface according to the rotation parameters set by the user, and export the rotating video of the 3D diagram C based on the export instruction input by the user, which is convenient for the user to observe the tissue and lesion offline later.

Step 5: generating a 2D schematic diagram C' corresponding to at least one target section by the processor 10 based on the 3D contour of the target tissue and the 3D contour of the target lesion, as shown in FIG. 6. The 2D schematic diagram C' corresponding to the target section includes: a target tissue graphic A' for representing the morphology of the target tissue and a target lesion graphic B' for presenting the morphology and location of the target lesion. It can be seen that the 2D schematic diagram C' is equivalent to a 2D schematic diagram of the lesion distribution. In this embodiment, multiple (two or more) 2D schematic diagrams C' corresponding to the target sections are generated to facilitate understanding the distribution of the lesion from different angles. The target section is a anatomical plane of the target tissue. For example, the type of the target section is one of the sagittal plane, transverse plane, and coronal plane. This embodiment is illustrated with FIG. 6 as an example, which has three 2D schematic diagrams C' corresponding to the target sections. These three target sections are the sagittal plane, transverse plane, and coronal plane of the target tissue (e.g., the uterus). After the 2D schematic diagrams C' corresponding to these target sections are displayed, users can quickly know which tissue organ the target lesion graphic B' belongs to and approximately where the target lesion is located in the target tissue. Moreover, the sagittal plane, transverse plane, and coronal plane are mutually perpendicular, which is equivalent to obtaining the three views of the target tissue and lesion, allowing for a very clear and comprehensive understanding of the lesion distribution.

There are various ways to generate the 2D schematic diagrams based on the 3D contours. Several of them are listed and explained below.

In one approach, the processor 10 maps the 3D contour of the target tissue onto at least one first target anatomical plane of the target tissue, obtaining the target tissue graphic A' in the 2D schematic diagram C' corresponding to the at least one first target section. The target tissue graphic A' is specifically used to present the morphology of the target tissue on the corresponding first target section. The at least one first target section includes one or more of the sagittal plane, transverse plane, and coronal plane, that is, the first target section may be the aforementioned target section. In this embodiment, the 3D contour of the uterus is mapped onto the sagittal plane of the uterus, obtaining the uterine graphic in the 2D schematic diagram corresponding to the sagittal plane, as shown in FIG. 6. This uterine graphic reflects the morphology of the uterus on the sagittal plane, that is, the contour of the uterus on the sagittal plane. When a user sees this uterine graphic, it may be known that the 2D schematic diagram corresponds to the sagittal plane and the target tissue is the uterus. The same applies to mapping the 3D contour of the uterus onto the transverse plane of the uterus to obtain the uterine graphic in the 2D schematic diagram corresponding to the transverse plane, and mapping the 3D contour of the uterus onto the coronal plane of the uterus to obtain the uterine graphic in the 2D schematic diagram corresponding to the coronal plane. The details thereof is similar to the above description, and will not be repeated here. In this embodiment, the target tissue graphic A' in the 2D schematic diagram C' includes the 2D contours of multiple major anatomical structures of the target tissue (as shown by a1' and a2' in the figure), allowing the user to better determine the current type of target tissue and the location of the lesion within the tissue.

Since the 2D schematic diagram C' is mainly used to represent the distribution of the lesion in the tissue, the target tissue graphic A' in it is basically the background of the target lesion graphic B', which is used to show the positional relationship of the lesion and represent the type of tissue. There are various methods to obtain the target tissue graphic A' in the 2D schematic diagram C' through mapping. Two methods are listed below for illustration.

In a first method, the processor 10 slices the 3D contour of the target tissue with at least one first target section. The number of first target sections determines the number of times the 3D contour of the target tissue is sliced. The 2D contour of the target tissue on the at least one first target section is obtained. The volumetric data is known, and the 3D contour of the target tissue corresponds to the volumetric data. The first target section of the target tissue is the first target section of the 3D contour of the target tissue, that is, the spatial relationship between the 3D contour of the target tissue and each first target section is known. Therefore, it is very convenient to obtain the 2D contour of the 3D contour of the target tissue on each first target section. Possibly in step 2, the 2D contour of the target tissue on multiple slices is obtained through automatic segmentation of multiple 2D ultrasound images or manual marking by the user. Then, the 2D contour on the first target section can be found. In summary, after obtaining the 2D contour, the processor 10 takes the 2D contour of the target tissue on the at least one first target section as the target tissue graphic in the corresponding 2D schematic diagram. For example, the 2D contour of the uterus on the sagittal plane is taken as the uterus graphic in the 2D schematic diagram corresponding to the sagittal plane, the 2D contour of the uterus on the transverse plane is taken as the uterus graphic in the 2D schematic diagram corresponding to the transverse plane, and the 2D contour of the uterus on the coronal plane is taken as the uterus graphic in the 2D schematic diagram corresponding to the coronal plane.

In a second method, the processor 10 projects the 3D contour of the target tissue onto the at least one first target section to obtain the target tissue graphic A' in the 2D schematic diagram C' corresponding to the at least one first target section. For the sagittal plane, transverse plane and coronal plane, the 2D contour obtained by slicing and projection is basically the contour with a larger area in the corresponding direction of the tissue. Therefore, both can well reflect the morphological characteristics of the major anatomical structure of the tissue.

The processor 10 maps the 3D contour of the target lesion onto at least one second target section of the target lesion to obtain at least one target lesion graphic. Similarly, the processor 10 can use the at least one second target section to section the 3D contour of the target lesion. The number of second target sections determines the number of times the 3D contour of the target lesion is sectioned, thereby obtaining the 2D contour of the target lesion on the at least one second target section, or the processor 10 projects the 3D contour of the target lesion onto the at least one second target section to obtain the 2D contour of the target lesion on the at least one second target section. The specific methods may be the same as those for mapping the target tissue, and will not be repeated here. Then, the processor 10 takes the 2D contour of the target lesion on the at least one second target section as the target lesion graphic.

The at least one second target section may be three sections that reflect the largest area of the target lesion in three perpendicular directions. If the target lesion is an ellipsoid, then these three second target sections are the sections containing the long and wide dimensions of the ellipsoid, the sections containing the long and thick dimensions, and the sections containing the thick and wide dimensions. That is, the target lesion graphic in each 2D schematic diagram may specifically be used to present two of the three dimensions of the target lesion, namely, length, width, and thickness. Since the situation where the lesion is located at the center of the tissue and has a very "correct" orientation is rare, the second target section of the lesion is usually not parallel to the first target section of the tissue. Of course, in a few cases, they may be parallel. That is, the 2D schematic diagram corresponding to the uterine sagittal plane usually cannot be obtained by directly mapping or projecting the 3D schematic diagram onto the uterine sagittal plane, but rather, the uterine graphic on the uterine sagittal plane and the myoma graphic reflecting the length and width, length and thickness, or thickness and width of the lesion need to be obtained first, and then the two are combined to form the 2D schematic diagram corresponding to the uterine sagittal plane.

The processor 10 fuses each of the at least one target lesion graphic into the target tissue graphic corresponding to the at least one first target section, thereby obtaining the 2D schematic diagram corresponding to the at least one first target section. For example, multiple target lesion graphics corresponding to the second target sections and multiple target tissue graphics corresponding to the first target sections are obtained. The processor 10 superimposes the target lesion graphic of each second target section onto the target tissue graphic of the corresponding first target section one by one, thereby obtaining the 2D schematic diagram corresponding to the first target section. Which target lesion graphic of the second target section is superimposed onto which target tissue graphic of the first target section may be predetermined. For example, the processor 10 fuses (such as superimposes) the target lesion graphic of each second target section onto the target tissue graphic of the corresponding first target section according to the predetermined correspondence between the second target section and the first target section, thereby obtaining the 2D schematic diagram of each first target section.

In another approach, the processor 10 maps the 3D contour of the target tissue onto at least one first target anatomical plane of the target tissue to obtain the target tissue graphic in the 2D schematic diagram corresponding to the at least one first target section. The specific process is the same as the previous approach and will not be repeated here. The main difference between this approach and the previous one lies in the generation method of the target lesion graphic. In this approach, the processor 10 obtains the size of the target lesion based on the 3D contour of the target lesion and generates at least one target lesion graphic according to the size of the target lesion. The way the processor 10 obtains the size of the target lesion is introduced in step 3 and will not be repeated here. The size of the target lesion may be multiple of the length, width and thickness. According to the length, width and thickness of the lesion, multiple target lesion graphics may be generated. In this embodiment, the processor 10 generates three target lesion graphics based on the length, width and thickness of the lesion. Specifically, an ellipse is generated with the length and width as the long and short axes respectively as the target lesion graphic in one 2D schematic diagram, an ellipse is generated with the length and thickness as the long and short axes respectively as the target lesion graphic in another 2D schematic diagram, and an ellipse is generated with the width and thickness as the long and short axes respectively as the target lesion graphic in yet another 2D schematic diagram. These target lesion graphics may be respectively fused onto multiple target tissue graphics to obtain the 2D schematic diagrams of multiple target sections. That is, the processor 10 fuses the at least one target lesion graphic respectively onto the target tissue graphic in the 2D schematic diagram corresponding to the at least one first target section to obtain the 2D schematic diagram corresponding to the at least one first target section. Similarly, which target lesion graphic is superimposed onto which target tissue graphic of the first target section may be predetermined. For example, the processor 10 fuses (such as superimposes) multiple target lesion graphics of the same target lesion onto the target tissue graphics of the corresponding first target sections respectively according to the preset correspondence between the length, width and thickness and the first target sections, thereby obtaining the 2D schematic diagrams of each first target section.

In some embodiments, it may also include step 6: the processor 10 displaying the 3D schematic diagram and/or the 2D schematic diagram corresponding to the at least one target section on the display interface of the display. This embodiment takes the display of the 3D schematic diagram and the 2D schematic diagram as an example for illustration. Each 2D schematic diagram and the 3D schematic diagram may be displayed centrally, as shown in FIG. 6. The four schematic diagrams are displayed in a four-grid form. The combination of 3D and 2D may present the location distribution and morphology of the lesion in the tissue very intuitively. It is convenient for clinical doctors to exchange the distribution of the lesion and is beneficial for non-ultrasound doctors, patients and their family to understand the condition.

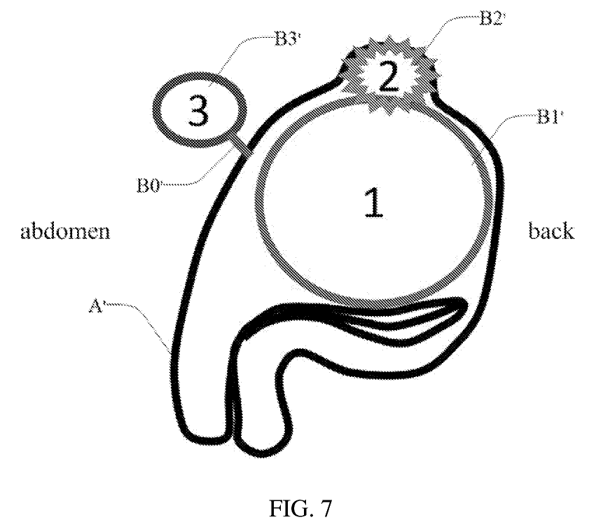

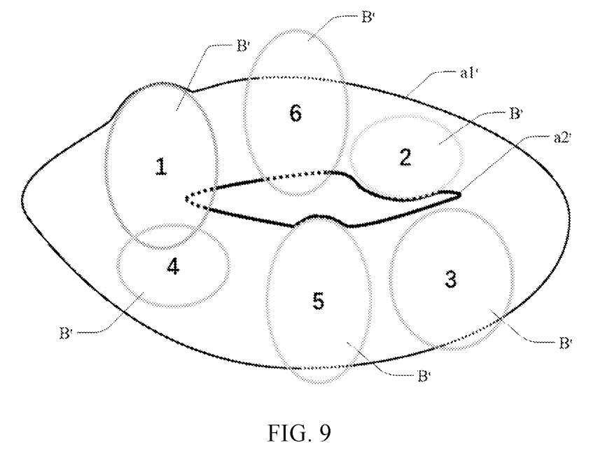

The drawing methods of 2D schematic diagrams may be diverse. Since they mainly include the contours of tissues and lesions, the contours of tissues and lesions may be drawn with lines, as shown in FIGS. 7 and 9. On this basis, different colors of lines may be used to draw different lesions and major anatomical structures. Internal textures may also be added to distinguish different anatomical structures and lesions, etc.

In some embodiments, as shown in FIG. 7, the 2D schematic diagram of the target section also includes a lesion pedicle graphic B0', which is obtained by mapping the 3D contour of the lesion pedicle onto the target section. The specific mapping method may be found in the relevant content of mapping the 3D contour of the target tissue/target lesion to the first/second target section, and will not be repeated here. Similarly, the target tissue graphic in the 2D schematic diagram includes the 2D contours of multiple major anatomical structures of the target tissue (such as a1', a2', etc.); and the point, on the 2D contour of the major anatomical structure adjacent to the target lesion graphic, that is closest to the target lesion graphic is the 2D attachment point of the lesion pedicle, that is, if the target lesion graphic is connected to the 2D contour of the adjacent major anatomical structure with the shortest line segment, the attachment point on the 2D contour of the major anatomical structure is the 2D attachment point of the lesion pedicle.

In one embodiment, the location of the 3D attachment point of the lesion pedicle on the 3D schematic diagram on the display interface can be adjusted. For example, the processor 10 receives a user operation of selecting a attachment point on the 3D schematic diagram and then moving the attachment point via the input device of the human-machine interaction device, and updates the location of the attachment point to the location after the movement based on this operation. Similarly, in the 2D schematic diagram, the location of the 2D attachment point of the lesion pedicle may also be adjusted. For example, the processor 10 receives the operation of the user selecting a attachment point on the 2D schematic diagram and then moving the attachment point through the input device of the human-machine interaction device, and updates the location of the attachment point to the location after the movement based on this operation.

In some embodiments, the appearance of the target lesion graphic in the 2D schematic diagram can reflect the benign or malignant nature of the lesion, and even the degree of malignancy. For example, a certain type of target lesion is pre-corresponded to a certain appearance of the target lesion graphic. The appearance of the target lesion graphic may refer to the roughness of its contour. For instance, benign lesions correspond to smooth contours (as shown in lesions 1 and 3 in FIG. 7), while malignant lesions correspond to rough contours (as shown in lesion 2 in FIG. 7). The higher the degree of malignancy, the rougher the contour. In the 2D schematic diagram of the uterine scene, the target lesion graphic of a benign fibroid is a smooth ellipse, and that of a malignant fibroid is an ellipse with spicules. The processor 10 then determines the appearance of the target lesion graphic based on the type of the target lesion. After the subsequent 2D schematic diagram is displayed, it allows users to know the type of the lesion by observing the roughness of the contour of the target lesion graphic, which is very intuitive. Of course, in addition to the roughness of the target lesion graphic, other distinctive appearance designs may also be used to achieve the purpose of differentiating lesion types, and this is not limited here.

In order to better reflect the positional differences among various major anatomical structures and target lesions, in the 2D schematic diagram, the 2D contours of each major anatomical structure and the target lesion graphics can be distinguished by different markers. The markers may be characters (such as text, letter codes, numbers, etc.) or colors, as long as they can be distinguished. The markers for the same lesion in the 3D schematic diagram and the 2D schematic diagram are the same, and the markers for the same major anatomical structure in the 3D schematic diagram and the 2D schematic diagram are also the same. This makes it convenient to match the 2D and 3D schematic diagrams. In this embodiment, different colors are used in the 2D schematic diagram to distinguish various major anatomical structures and target lesions.

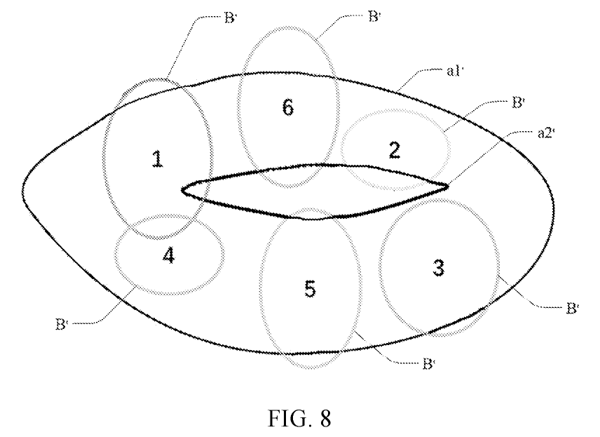

When the target lesion graphic and the target tissue graphic are fused and displayed in the 2D schematic diagram, there may be overlapping and/or mutual squeezing of the spatial locations of the lesion and the tissue. This can be distinguished through differentiated representation. Specifically, the processor 10 acquires the spatial location relationship between the target lesion and the major anatomical structure. If the spatial location relationship between the target lesion and the major anatomical structure has been obtained in the previous steps, it may be used directly. Of course, it may also receive the spatial relationship between the target lesion and the major anatomical structure determined by the user, that is, a user can input or adjust the spatial location relationship between the target lesion and the major anatomical structure. When there is an overlap of the 2D contour of the target lesion and the major anatomical structure in the 2D schematic diagram (as shown by lesions 1, 2-6 in FIG. 8), and it is determined based on the spatial location relationship between the target lesion and the major anatomical structure that the two do not contact, that is, if the 2D contour of the target lesion and the major anatomical structure overlap in the 2D schematic diagram, it indicates that either there is an occlusion relationship or the lesion has squeezed the major anatomical structure (squeezing relationship). Then, through the spatial location relationship between the two, it may be determined whether they are in an occlusion or squeezing relationship. Occlusion means that the two do not have direct contact. In this case, the processor 10 can perform the first differentiated representation on the part of the 2D contour of the major anatomical structure in the 2D schematic diagram that overlaps with the target lesion graphic to present that the target lesion and the major anatomical structure do not contact.

When, in the 2D schematic diagram, there is an overlap between the 2D contour of the target lesion and that of the major anatomical structure, and it is determined that the two are in contact based on their spatial positional relationship, the spatial contact is indicated by the overlap in two dimensions, suggesting a squeezing relationship. The processor 10 can perform a second differentiated representation on the part of the 2D contour of the major anatomical structure in the 2D schematic diagram that overlaps with the target lesion graphic, to represent the contact and squeezing between the target lesion and the major anatomical structure. Clearly, the first differentiated representation is different from the second differentiated representation.

As shown in FIG. 9, the first differentiated representation may be to blur the parts of the 2D contour of the major anatomical structure that overlap with the lesion graphics, such as representing the overlapping 2D contour with a dotted line, as shown by the dotted lines in the target lesion graphics 1, 3, 5 and 6 in the figure. Of course, the parts of the target lesion graphics that overlap with the major anatomical structure may also be blurred, but this is not conducive to highlighting the target lesion graphics. The second differentiated representation may be to adjust the parts of the 2D contour of the major anatomical structure that overlap with the target lesion graphics, so that the 2D contour of the major anatomical structure and the target lesion graphics that overlap are adjacent and similar. This may be achieved by re-interpolating and redrawing the 2D contour of the overlapping region of the target lesion graphics on the target tissue graphics, or by calculating the overlapping region of the target lesion graphics and the target tissue graphics and redrawing the contour of that region. The adjusted target tissue graphics are shown in FIG. 9. The target lesion graphics 1, 2 and 5 have a section of contour adjacent to the contour of the target tissue graphics. At the adjacent location, the target tissue graphics has undergone deformation, indicating that the two are in a squeezing relationship. Of course, the first and second differentiated representations may also be implemented in other ways, as long as the differences between the two are presented. It can be seen that the processor 10 can automatically identify and visually and intuitively present the overlapping and squeezing positional relationships between tissues and lesions on a 2D schematic diagram, demonstrating the automation and intelligence of lesion distribution presentation.

In some embodiments, the processor 10 can also display the patient orientation indicators corresponding to the 3D schematic diagram on the display interface of the display. The displayed patient orientation indicators may be at least one of left, right, abdomen, back, head, and tail. Similarly, as shown in FIG. 7, the processor 10 can also display the corresponding patient orientation on the 2D schematic diagram of the target section according to the type of the target section; the displayed patient orientation may be at least one of left, right, abdomen, back, head, and tail. That is, the type of the target section may be divided into three types: sagittal plane, transverse plane, and coronal plane. The patient orientation may be divided into six types: left, right, abdomen, back, head, and tail, among which two are opposite. The patient orientation can well indicate the relative position relationship between the target tissue graphic and the body of a patient. The patient orientation corresponding to the sagittal plane may be abdomen and back, which may represent the relative position relationship between the abdomen and back of the patient and the target tissue graphic. The patient orientation corresponding to the coronal plane may be head and tail, and the patient orientation corresponding to the transverse plane may be left and right. Of course, more patient orientations may also be presented.

When the processor 10 identifies multiple target lesions from the volumetric data, it can number the 3D contours of each target lesion in the 3D schematic diagram, or number the target lesion graphics in the 2D schematic diagram corresponding to the target section, as shown in FIGS. 7-9. Each lesion may be numbered with a digit. Users can also manually change the lesion numbers via input devices (such as knobs, keyboards, function keys, etc.).

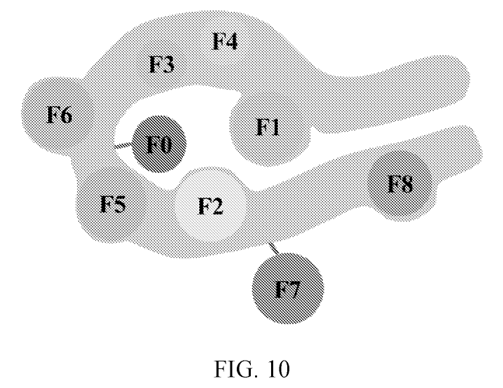

As shown in FIG. 10, the display interface of the display can also display a tumor classification reference atlas to prompt a user to classify a tumor type for the target lesion. The tumor classification reference atlas includes schematic graphical representations of a plurality of different tumor types (such as F0-F8 in the figure), each of which has image features for representing the tumor type. The image features of different tumor types are different, that is, the tumor type is uniquely identified by the image features. The image features in the figure include typing markers (F0-F8) and colors, both of which may uniquely identify the tumor type. For the uterus, the tumor classification reference atlas can follow the regulations of the International Federation of Gynecology and Obstetrics (FIGO), dividing fibroids into 9 types, where F0-F8 represent FIGO types 0-8. Each tumor schematic graphic also reflects the relative positional relationship between the fibroid and the endometrium and the uterine wall.

Users can refer to the tumor classification reference atlas to classify the target lesion. For example, they may input or select the tumor type, and each tumor type is pre-associated with image features. The processor 10 determines the tumor type of the lesion based on a user classification operation, and then may display the image features of the determined tumor type (such as typing markers and corresponding colors) on the 3D contour of the target lesion in the 3D schematic diagram, or can display the image features of the determined tumor type (such as corresponding typing markers and/or colors, etc.) on the target lesion graphic in the 2D schematic diagram corresponding to the target section.

The International Federation of Gynecology and Obstetrics (FIGO) classifies fibroids based on their relative position to the endometrium and the uterine wall. Therefore, the processor 10 can obtain the location of the target lesion in the target tissue (as described above), determine the tumor type of the target lesion according to its location in the target tissue based on a predetermined classification rule, and then display the image features of the determined tumor type on the 3D contour of the target lesion in the 3D schematic diagram, and/or on the target lesion graphic in the 2D schematic diagram of the target section.

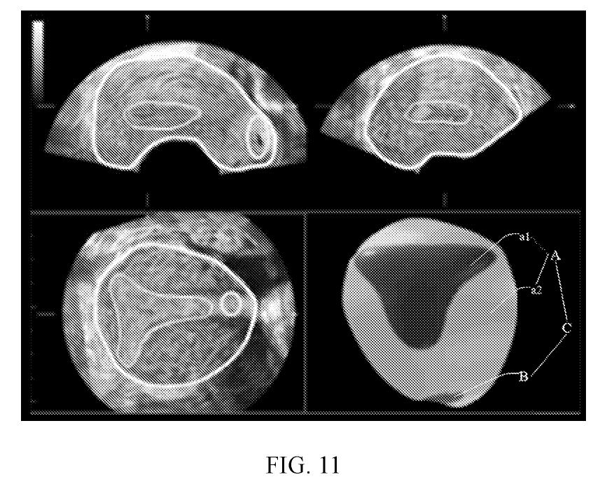

In some embodiments, the processor can also obtain at least two mutually perpendicular 2D ultrasound images from the ultrasound volumetric data, as shown in FIG. 11. In this embodiment, the at least two mutually perpendicular 2D ultrasound images are 2D ultrasound images of three perpendicular planes. For example, a spatial coordinate system is established with the 3D schematic diagram as the center. These three perpendicular planes may be the three planes X0Y, X0Z, and Y0Z of the spatial coordinate system. The 3D schematic diagram corresponds to the ultrasound volumetric data. Therefore, the processor 10 can obtain the 2D ultrasound images of the X0Y, X0Z, and Y0Z planes from the ultrasound volumetric data based on the correspondence between the 3D schematic diagram and the ultrasound volumetric data. The processor 10 marks the contour of the target tissue in the at least two mutually perpendicular 2D ultrasound images (as shown by the white lines in FIG. 11). If the at least two mutually perpendicular 2D ultrasound images contain the target lesion, the contour of the target lesion is also marked (as shown by the yellow lines in FIG. 11). Then, the 3D schematic diagram C and the at least two mutually perpendicular 2D ultrasound images are displayed on the display interface of the display. In some embodiments, the 3D schematic diagram C may be continuously displayed, but the at least two mutually perpendicular 2D ultrasound images and the 2D schematic diagrams corresponding to each target section do not need to be displayed simultaneously. They can be switched. For example, the current display interface shows the 2D schematic diagrams corresponding to each target section and the 3D schematic diagram C, as shown in FIG. 6. The processor 10 receives a switching instruction. In response to the switching instruction, the processor 10 switches the 2D schematic diagrams corresponding to each target section displayed on the display interface to the at least two mutually perpendicular 2D ultrasound images, that is, from FIG. 6 to FIG. 11. If the processor 10 receives another switching instruction, it switches the at least two mutually perpendicular 2D ultrasound images displayed on the display interface to the 2D schematic diagrams corresponding to each target section, that is, from FIG. 11 to FIG. 6.

When the content of FIG. 11 is displayed on the display interface, the 3D schematic diagram C is displayed in conjunction with (i.e., displayed in a linked manner) at least two mutually perpendicular 2D ultrasound images. The processor 10 receives an instruction for rotating the 3D schematic diagram C. In response to this instruction for rotating the 3D schematic diagram C, the processor 10 rotates the 3D schematic diagram C displayed on the display interface. When the 3D schematic diagram C changes its orientation, its contours on the X0Y, X0Z, and Y0Z planes also change. Therefore, the at least two mutually perpendicular 2D ultrasound images need to be updated. Thus, the processor 10 re-obtains the at least two mutually perpendicular 2D ultrasound images that have changed due to the rotation of the 3D schematic diagram C from the ultrasound volumetric data, that is, repeats the steps of the previous paragraph to obtain new 2D ultrasound images, thereby updating the at least two mutually perpendicular 2D ultrasound images. In this way, the user can rotate the 3D schematic diagram C to view the lesion they want to see, and the corresponding three perpendicular 2D ultrasound images will also be updated in conjunction. It is equivalent to the user being able to see the ultrasound image of any section by rotating the 3D schematic diagram C, which is very convenient.

If a user is not satisfied with the generated 3D schematic diagram and 2D schematic diagram, editing operations such as adding, deleting, modifying, and regenerating can by performed by the user. This also includes editing the size, direction, and location of the lesion. For example, the processor 10 receives an instruction for regenerating the schematic diagram. In response to this instruction, the method for presenting the lesion distribution is re-executed, that is, the various steps shown in FIG. 2 are re-executed, thereby regenerating and displaying the 3D schematic diagram and the 2D schematic diagram corresponding to the target section.

Since the contours of the target tissue and the target lesion in the 2D ultrasound image shown in FIG. 11 are derived from the ultrasound volumetric data, the 3D contours of the tissue and the lesion may be generated based on the 2D contours in the ultrasound image (see step 2 for details), or there is a corresponding relationship between these 3D contours and the 2D contours in the ultrasound image. Therefore, adjusting the contours of the target tissue and the target lesion in the 2D ultrasound image may adjust the 3D contours of the tissue and the lesion, that is, adjust the 3D schematic diagram. For example, the processor 10 receives an instruction for adjusting the contour of the target tissue in the 2D ultrasound image, and in response to the instruction, adjusts the contour of the target tissue in the 2D ultrasound image; and adjusts the 3D contour of the target tissue based on the adjusted contour of the target tissue in the 2D ultrasound image, thereby updating the 3D schematic diagram. In this way, only one adjustment of the contour in the 2D ultrasound image is needed to adjust both the 2D and 3D schematic diagrams, which is very convenient. Similarly, the processor 10 can receive an instruction for adjusting the contour of the target lesion in the 2D ultrasound image, and in response to the instruction, adjust the contour of the target lesion in the 2D ultrasound image; and adjust the 3D contour of the target lesion based on the adjusted contour of the target lesion in the 2D ultrasound image, thereby updating the 3D schematic diagram. Adjusting the contour of the target lesion is equivalent to adjusting the shape and size of the target lesion. Users can also adjust the location and/or direction of the target lesion, that is, the orientation of the target lesion. For example, the processor 10 can receive an instruction for adjusting the location and/or direction of the target lesion in the 2D ultrasound image, and in response to the instruction, adjust the location and/or direction of the target lesion in the 2D ultrasound image; and adjust the location and/or direction of the 3D contour of the target lesion based on the adjusted location and/or direction of the target lesion in the 2D ultrasound image, thereby updating the 3D schematic diagram.

Similarly, users can also delete lesions in 2D ultrasound images. For instance, users can select the target lesion to be deleted, either in the 2D ultrasound image or in the 3D schematic diagram. The processor 10 receives the instruction for deleting the selected target lesion and, in response to this instruction, deletes the contour marker of the selected target lesion in the 2D ultrasound image and/or the 3D contour of the selected target lesion in the 3D schematic diagram.

From the above content, it can be known that the present disclosure is based on 3D ultrasound images to obtain 3D structure diagrams of tissues and lesions, and can map and generate corresponding 2D diagrams. The specific process is to obtain 3D volumetric data containing the target tissue and target lesion, and automatically/semi-automatically generate the 3D contour of the target tissue and the 3D contour of the target lesion based on the 3D volumetric data. These 3D contours are displayed together according to their positional relationship (3D diagram) to present the tissues and lesions in 3D space. The 3D contours of the tissues and lesions are automatically mapped to the 2D sagittal plane, transverse plane, and coronal plane to generate 2D diagrams corresponding to these sections, presenting the 2D structure and lesion distribution of the tissues and lesions. Doctors can quickly understand and convey the location and distribution of the lesions with the help of 3D and 2D diagrams, without having to laboriously view ultrasound images and manually draw diagrams, improving the efficiency of conveying the distribution of lesions among doctors.

The present disclosure refers to various exemplary embodiments for illustrative purposes. However, those skilled in the art will recognize that modifications and alterations may be made to these embodiments without departing from the scope of the disclosure. For instance, individual operational steps and components for performing such steps may be implemented in diverse manners depending on specific applications or considerations of cost functions associated with system operations (e.g., one or more steps may be deleted, modified, or consolidated with other steps).

Moreover, as understood by those skilled in the art, the principles disclosed may be embodied in a computer program product stored on a non-transitory computer-readable storage medium preloaded with computer-readable program code. Any tangible, non-transitory computer-readable storage medium may be utilized, including but not limited to: magnetic storage devices (e.g., hard disks, floppy disks); optical storage devices (e.g., CD-ROMs, DVDs, Blu-ray discs); and flash memory devices. The computer program instructions may be loaded onto a general-purpose computer, special-purpose computer, or other programmable data processing apparatus to create a machine, such that the instructions executed on the computer or programmable apparatus produce means for implementing specified functions. These instructions may also reside in a computer-readable memory, directing the computer or programmable apparatus to operate in a defined manner, thereby forming an article of manufacture comprising functional implementation means. Furthermore, the computer program instructions may be executed on a computer or programmable data processing apparatus to generate a computer-implemented process, wherein the executed instructions provide steps for realizing the specified functionality, including but not limited to: technical improvements in data processing efficiency (e.g., optimized memory allocation); and enhanced accuracy in algorithmic execution (e.g., reduced error margins in machine learning models).

While the principles disclosed herein have been illustrated through various embodiments, it should be understood that structural configurations, material selections, and component proportions particularly suited to specific operational environments may be modified without departing from the scope and spirit of the disclosure. Such modifications, along with other adaptations or adjustments, shall be encompassed within the scope of the present disclosure.

The foregoing detailed description has been described with reference to various embodiments. However, those skilled in the art will recognize that modifications and variations may be made without departing from the scope of the disclosure. Accordingly, the description of the disclosure shall be interpreted in an illustrative rather than restrictive sense, and all such modifications are intended to be included within its scope. Similarly, discussions of advantages, alternative solutions to problems, and operational benefits associated with the embodiments are provided above. Nevertheless, benefits, advantages, solutions to problems, and any elements that may produce such effects or render them more explicit shall not be construed as critical, required, or essential. Furthermore, the term 'coupled' and its derivatives encompass physical connections (e.g., mechanical joints), electrical connections (e.g., circuit interconnects), magnetic linkages (e.g., inductive coupling), optical interfaces (e.g., fiber-optic alignment), communication channels (e.g., wireless protocols), functional integrations (e.g., software APIs), and any other form of association that achieves operational interaction.

Those skilled in the art will recognize that numerous modifications to the details of the above-described embodiments may be made without departing from the fundamental principles of the disclosed subject matter. Accordingly, the scope of the present disclosure shall be determined solely by the claimsand their legal equivalents.

1. An ultrasound imaging apparatus, comprising:
an ultrasound probe, configured to transmit ultrasound waves and receive corresponding ultrasound echoes;
a transmit and receive control circuit, configured to control the ultrasound probe to transmit the ultrasound waves and receive the ultrasound echoes; and
a processor, configured to:
obtain volumetric data containing a target tissue and a target lesion;
identify the target tissue from the volumetric data, and generate a three-dimensional contour of the target tissue;
identify the target lesion from the volumetric data, and generate a three-dimensional contour of the target lesion;
generate a three-dimensional schematic diagram based on the three-dimensional contour of the target tissue and the three-dimensional contour of the target lesion, wherein the three-dimensional schematic diagram is configured to represent a morphology of the target tissue, and a morphology and a location of the target lesion in the target tissue; and
generate a two-dimensional schematic diagram corresponding to at least one target section based on the three-dimensional contour of the target tissue and the three-dimensional contour of the target lesion, wherein the two-dimensional schematic diagram corresponding to the target section comprises: a target tissue graphic configured to represent the morphology of the target tissue, and a target lesion graphic configured to represent the morphology and location of the target lesion; and the target section comprises an anatomical plane of the target tissue.

2. The ultrasound imaging apparatus according to claim 1, wherein the processor is further configured to:
display the three-dimensional schematic diagram and/or the two-dimensional schematic diagram corresponding to at least one target section on a display interface of a display.

3. The ultrasound imaging apparatus according to claim 1, wherein the volumetric data comprises ultrasound volumetric data; and the processor is further configured to:
obtain, from the ultrasound volumetric data, two-dimensional ultrasound images corresponding to at least two mutually perpendicular sections;
mark a contour of the target tissue in the two-dimensional ultrasound images corresponding to at least two mutually perpendicular sections, and mark a contour of the target lesion when the two-dimensional ultrasound images corresponding to at least two mutually perpendicular sections contain the target lesion; and
display the three-dimensional schematic diagram and the two-dimensional ultrasound images corresponding to at least two mutually perpendicular sections on a display interface of a display.

4. The ultrasound imaging apparatus according to claim 3, wherein the processor is further configured to:
receive a switching instruction and, in response to the switching instruction, switch from the two-dimensional ultrasound images corresponding to at least two mutually perpendicular sections displayed on the display interface to the two-dimensional schematic diagram corresponding to at least one target section.

5. The ultrasound imaging apparatus according to claim 3, wherein the three-dimensional schematic diagram and the two-dimensional ultrasound images corresponding to at least two mutually perpendicular sections are capable of being displayed in a linked manner; and the processor is further configured to:
in response to an instruction for rotating the three-dimensional schematic diagram, rotate the three-dimensional schematic diagram displayed on the display interface;
re-acquire, from the ultrasound volumetric data, the two-dimensional ultrasound images corresponding to at least two mutually perpendicular sections that have changed due to the rotation of the three-dimensional schematic diagram; and
update the two-dimensional ultrasound images corresponding to at least two mutually perpendicular sections.

6. The ultrasound imaging apparatus according to claim 1, wherein the processor is further configured to:
identify a type of the target lesion in the volumetric data, or, receive a user-selected type and determine the user-selected type as the type of the target lesion in the volumetric data, wherein the type of the target lesion comprises at least benign and malignant;
determine an appearance of the three-dimensional contour of the target lesion based on the type of the target lesion, wherein an appearance for the three-dimensional contour of the target lesion is predefined for each type; and/or, determine an appearance of the target lesion graphic in the two-dimensional schematic diagram based on the type of the target lesion, wherein an appearance for the target lesion graphic is predefined for each type.

7. The ultrasound imaging apparatus according to claim 1, wherein the three-dimensional schematic diagram comprises three-dimensional contours of a plurality of major anatomical structures of the target tissue and the three-dimensional contour of the target lesion;
the target tissue graphic in the two-dimensional schematic diagram comprises two-dimensional contours of the plurality of major anatomical structures of the target tissue;
in the three-dimensional schematic diagram, the three-dimensional contour of each major anatomical structure and the three-dimensional contour of the target lesion are distinguished by different markers; and/or, in the two-dimensional schematic diagram, the two-dimensional contour of each major anatomical structure and the target lesion graphic are distinguished by different markers.

8. The ultrasound imaging apparatus according to claim 7, wherein the processor is further configured to:
obtain a spatial relationship between the plurality of major anatomical structures;
determine, based on the spatial relationship between the plurality of major anatomical structures, whether one major anatomical structure is located internally to another major anatomical structure; and
if so, render the three-dimensional contour of said another major anatomical structure transparent or semi-transparent in the three-dimensional schematic diagram.

9. The ultrasound imaging apparatus according to claim 7, wherein when a plurality of target lesions are identified from the volumetric data, the three-dimensional contours of the target lesions in the three-dimensional schematic diagram are numbered, and/or, the target lesion graphics in the two-dimensional schematic diagram corresponding to the target section are numbered.

10. The ultrasound imaging apparatus according to claim 2, wherein
the display interface is further configured to display a tumor classification reference atlas to prompt a user to classify a tumor type for the target lesion, wherein the tumor classification reference atlas comprises schematic graphical representations of a plurality of different tumor types, each schematic graphical representation has an image feature indicative of a tumor type, and the image features of different tumor types are different;
the three-dimensional schematic diagram comprises a three-dimensional contour of the target lesion;
the processor is further configured to:
determine a tumor type of the target lesion based on a classification operation performed by the user; and
display an image feature of the determined tumor type on the three-dimensional contour of the target lesion in the three-dimensional schematic diagram, and/or, display an image feature of the determined tumor type on the target lesion graphic in the two-dimensional schematic diagram corresponding to the target section.

11. The ultrasound imaging apparatus according to claim 2, wherein
the display interface is further configured to display a tumor classification reference atlas to prompt a user to classify a tumor type for the target lesion, wherein the tumor classification reference atlas comprises schematic graphical representations of a plurality of different tumor types, each schematic graphical representation has an image feature indicative of a tumor type, and the image features of different tumor types are different;
the three-dimensional schematic diagram comprises a three-dimensional contour of the target lesion;
the processor is further configured to:
obtain the location of the target lesion in the target tissue;
determine a tumor type of the target lesion according to a predetermined classification rule using the location of the target lesion in the target tissue; and
display the image features of the determined tumor type on the three-dimensional contour of the target lesion in the three-dimensional schematic diagram, and/or, display the image features of the determined tumor type on the target lesion graphic in the two-dimensional schematic diagram corresponding to the target section.

12. The ultrasound imaging apparatus according to claim 1, wherein the three-dimensional schematic diagram comprises three-dimensional contours of a plurality of major anatomical structures of the target tissue and the three-dimensional contour of the target lesion; the target tissue graphic in the two-dimensional schematic diagram comprises two-dimensional contours of the plurality of major anatomical structures of the target tissue; and the processor is further configured to:
obtain a spatial relationship between the target lesion and the major anatomical structures;
when, within the two-dimensional schematic diagram, the target lesion graphic overlaps a two-dimensional contour of a major anatomical structure, and it is determined based on the spatial relationship between the target lesion and said major anatomical structure that non-contact exists therebetween, perform a first differentiated representation on a portion of the two-dimensional contour of said major anatomical structure in the two-dimensional schematic diagram that overlaps with the target lesion graphic, thereby indicating non-contact between the target lesion and the major anatomical structure; and
when, within the two-dimensional schematic diagram, the target lesion graphic overlaps a two-dimensional contour of a major anatomical structure, and it is determined based on the spatial relationship between the target lesion and said major anatomical structure that contact exists therebetween, perform a second differentiated representation on a portion of the two-dimensional contour of said major anatomical structure in the two-dimensional schematic diagram that overlaps with the target lesion graphic, thereby indicating contact between the target lesion and the major anatomical structure and generating squeezing; wherein the first differentiated representation and the second differentiated representation are different.

13. The ultrasound imaging apparatus according to claim 1, wherein the processor is further configured to:
obtain a location of the target lesion in the target tissue; and
generate a three-dimensional contour of a lesion pedicle in the three-dimensional schematic diagram based on the location of the target lesion in the target tissue;
wherein the three-dimensional contour of the target tissue is connected to the three-dimensional contour of the target lesion via the three-dimensional contour of the lesion pedicle; and
the two-dimensional schematic diagram corresponding to the target section further comprises a lesion pedicle graphic that is obtained by projecting the three-dimensional contour of the lesion pedicle onto the target section.

14. The ultrasound imaging apparatus according to claim 13,
wherein, the three-dimensional schematic diagram comprises the three-dimensional contour of the target tissue and the three-dimensional contour of the target lesion; the three-dimensional contour of the target tissue comprises three-dimensional contours of a plurality of major anatomical structures of the target tissue; on the three-dimensional contour of a major anatomical structure adjacent to the three-dimensional contour of the target lesion, a point that is closest to the three-dimensional contour of the target lesion defines a three-dimensional attachment point of the lesion pedicle, and a location of the three-dimensional attachment point of the lesion pedicle is adjustable; and/or,
wherein, the target tissue graphic in the two-dimensional schematic diagram comprises two-dimensional contours of the plurality of major anatomical structures of the target tissue; on the two-dimensional contour of a major anatomical structure adjacent to the target lesion graphic, a point that is closest to the target lesion graphic defines a two-dimensional attachment point of the lesion pedicle; and a location of the two-dimensional attachment point of the lesion pedicle is adjustable.

15. The ultrasound imaging apparatus according to claim 2, wherein the processor is further configured to:
display a patient orientation indicator corresponding to the three-dimensional schematic diagram on the display interface of the display; and/or,
display a corresponding patient orientation on the two-dimensional schematic diagram corresponding to the target section based on a type of the target section, wherein the type of the target section is one of a sagittal plane, a transverse plane and a coronal plane.

16. The ultrasound imaging apparatus according to claim 3, wherein the processor is further configured to:
receive an instruction for adjusting the contour of the target tissue in the two-dimensional ultrasound images and, in response to said instruction, adjust the contour of the target tissue in the two-dimensional ultrasound images; and adjust the three-dimensional contour of the target tissue based on the adjusted contour of the target tissue, thereby updating the three-dimensional schematic diagram;
or, receive an instruction for adjusting the contour of the target lesion in the two-dimensional ultrasound images and, in response to said instruction, adjust the contour of the target lesion in the two-dimensional ultrasound images; and adjust the three-dimensional contour of the target lesion based on the adjusted contour of the target lesion, thereby updating the three-dimensional schematic diagram;
or, receive an instruction for deleting a selected target lesion and, in response to said instruction, delete a contour marker of the selected target lesion in the two-dimensional ultrasound images; and/or, delete a three-dimensional contour of the selected target lesion in the three-dimensional schematic diagram; wherein the selected target lesion is selected by a user in the two-dimensional ultrasound images or the three-dimensional schematic diagram.

17. The ultrasound imaging apparatus according to claim 1, wherein the processor is further configured to obtain a location of the target lesion in the target tissue; and
the processor being configured to generate the three-dimensional schematic diagram based on the three-dimensional contour of the target tissue and the three-dimensional contour of the target lesion comprises the processor being configured to:
fuse the three-dimensional contour of the target lesion into the three-dimensional contour of the target tissue based on the location of the target lesion in the target tissue, thereby obtaining the three-dimensional schematic diagram.

18. The ultrasound imaging apparatus according to claim 1, wherein the processor being configured to generate the two-dimensional schematic diagram corresponding to at least one target section based on the three-dimensional contour of the target tissue and the three-dimensional contour of the target lesion comprises the processor being configured to:
map the three-dimensional contour of the target tissue onto at least one first target section of the target tissue to obtain the target tissue graphic in the two-dimensional schematic diagram corresponding to the target section, wherein the target tissue graphic is configured to represent a morphology of the target tissue on a corresponding first target section; map the three-dimensional contour of the target lesion onto at least one second target section of the target lesion to obtain at least one target lesion graphic; and fuse the at least one target lesion graphic respectively into the target tissue graphic in the two-dimensional schematic diagram corresponding to the at least one first target section to obtain the at least one two-dimensional schematic diagram corresponding to the at least one first target section; wherein the at least one first target section comprises one or more of a sagittal plane, a transverse plane, and a coronal plane;
or, map the three-dimensional contour of the target tissue onto at least first target section of the target tissue to obtain the target tissue graphic in the two-dimensional schematic diagram corresponding to at least one target section; obtain a dimension of the target lesion based on the three-dimensional contour of the target lesion, and generate at least one target lesion graphic based on the dimension of the target lesion; and fuse at least one target lesion graphic respectively into the target tissue graphic in the two-dimensional schematic diagram corresponding to the at least one first target section to obtain at least one two-dimensional schematic diagram corresponding to the at least one first target section; wherein the at least one first target section comprises one or more of a sagittal plane, a transverse plane, and a coronal plane.

19. The ultrasound imaging apparatus according to claim 18, wherein the processor being configured to map the three-dimensional contour of the target tissue onto at least one first target section of the target tissue and obtain the target tissue graphic in the two-dimensional schematic diagram corresponding to the at least one first target section comprises the processor being configured to:
slice the three-dimensional contour of the target tissue with the at least one first target section to obtain a two-dimensional contour of the target tissue on the at least one first target section, and use the two-dimensional contour of the target tissue on the at least one first target section as the target tissue graphic in the corresponding two-dimensional schematic diagram;
or, project the three-dimensional contour of the target tissue onto the at least one first target section, and obtain the target tissue graphic in the two-dimensional schematic diagram corresponding to the at least one target section.

20. A method for presenting lesion distribution, comprising:
obtaining volumetric data containing a target tissue and a target lesion;
identifying the target tissue from the volumetric data, and generating a three-dimensional contour of the target tissue;
identifying the target lesion from the volumetric data, and generating a three-dimensional contour of the target lesion;
generating a three-dimensional schematic diagram based on the three-dimensional contour of the target tissue and the three-dimensional contour of the target lesion, wherein the three-dimensional schematic diagram is configured to represent a morphology of the target tissue, and a morphology and a location of the target lesion in the target tissue; and
generating a two-dimensional schematic diagram corresponding to at least one target section based on the three-dimensional contour of the target tissue and the three-dimensional contour of the target lesion, wherein the two-dimensional schematic diagram corresponding to the target section comprises: a target tissue graphic for representing the morphology of the target tissue, and a target lesion graphic for representing the morphology and location of the target lesion; the target section comprises an anatomical plane of the target tissue.
